(19) United States
(12) Patent Application Publication
(10) Pub. No.: US 2008/0268066 A1
(43) Pub. Date: Oct. 30, 2008

(54) Synergistic Formulation for Preventing and/or Treating Diabetes

(75) Inventors: Hongping Yie, Huaibei (CN); Zuolin Zhu, San Diego, CA (US); Meg M. Sun, San Diego, CA (US)

Correspondence Address: HAMRE, SCHUMANN, MUELLER & LARSON, P.C., P.O. BOX 2902, MINNEAPOLIS, MN, 55402-0902, US

(73) Assignee: PFICKER PHARMACEUTICALS LTD., Huaibei (CN)

(21) Appl. No.: 11/887,558

(22) PCT Filed: Sep. 23, 2005
(86) PCT No.: PCT/CN05/01555

§ 371 Date: Oct. 1, 2007

(30) Foreign Application Priority Data
Mar. 30, 2005 (CN) 200510062588.4

(51) Int. Cl. A61K 33/30; A61P 3/10

(52) U.S. Cl. 424/641

(57) ABSTRACT

The present invention discloses a composition preparation for preventing and treating diabetes, which mainly contains oral hypoglycemic agents, Vitamin E, chromic pyridine formate, selenium, lutein, folic acid, Vitamin C, alpha_lipoic acid, lycopene, nicotinamide, coenzyme Q10, and vitamin complexes and mineral. The medicament of the invention can decrease oxidative stress in vivo, promote body immunity, repair the critical organs in vivo such as pancreas, by supplying the mineral, vitamins and other substances which are insufficient in the patient suffering from diabetes, to prevent and treat diabetes.

### FIELD OF THE INVENTION

The invention belongs to the field of pharmaceutical preparation. specifically, a composition for preventing and/or treating diabetes.

### BACKGROUND ARTS

Diabetes may be classified into two types according to the deficiency of insulin: one is type I diabetes, which is caused by an absolute deficiency of insulin, while the other is type II diabetes, which is characterized in a relative deficiency of insulin or in compromised efficacy of insulin though being present at the normal level. Both types are associated with increased blood sugar.

Diabetes is refractory, lasting, it is life-time disease at current, and widespread. It is often associated with the complications such as systematic neuropathy, micro-vascular and major vascular diseases. With the changes in people's eating habits such as the uptake of excessive caloric and less exercise, more and more people get diabetes. The incidence of diabetes has been on rise in both the developed countries and the developing countries. Diabetes is one of the most health-threatening non-infectious diseases followed angiocardiopathy/cerebrovascular disease and cancer. In China, the diagnosed diabetes is about 50 million now, while 30 million more with light or insignificant symptoms may have been left out or delayed in diagnosis. In the United States, there is about 18.2 million patients with diabetes patients, about 6.3% of the whole population, wherein about 13 million are diagnosed, while about 5.2 million patients are not yet.

Diabetes being a chronic disease, the real damage is believed to be the endocrine dysfunction caused by the loss of a variety of essentials through the exuding of sugar in urine and the symptoms such as overdrinking, over-urinating, overeating, losing-weight, dizziness, hypodynamia and the like, which may further develops to cause various acute and chronic severe complications threatening to health and life.

After insulin was invented in 1921, many acute diabetic complications was no longer major fetal diseases, while the chronic ones became prominent and more threatening. The chronic diabetic complications are the results of a long period accumulation owing to not controlling the patients' blood sugar for a long time and the whole endocrine system being disturbance. The chronic diabetic complications are the main causes of the patients' deformity, life quality degressive and death. Whether the chronic diabetic complications occurs earlier or not and whether the chronic diabetic complications are more severe or not have the direct relationship with the blood sugar controlling, blood lipid and blood pressure. The chronic diabetic complications conclude diabetic fundus, diabetic renopathy, diabetic neuropathy, diabetic microangiopathy, diabetic pedopathy, diabetic dermatopathy, cancer, etc.

Diabetic fundus is one of the most popular chronic diabetic complications, which is also one of the most important reasons to arouse binocular blindness in the world. Diabetes can cause various opthalmopathy, such as corneal ulcer, glaucoma, vitreous hemorrhage, etc. But the most popular ones, which have the greatest impact, are diabetic retinopathy and cataract.

Diabetic renopathy is one of the severest chronic diabetic complications, which is also one of the main causes of death. It was reported that 50% of the patients with type I diabetes died of chronic renal failure and 5-10% of the patients with type II diabetes patients also died of chronic renal failure.

Diabetic neuropathy is one of the chronic diabetic complications with the highest incidence, since diabetes do great harm on nerve. Diabetic neuropathy can affect each part of the nervous system of human body, such as brain and spinal cord of central nervous system, cranial nerves of peripheral nervous system, esthetic and motorial peripheral nerve and vegetative nerve, etc. But among them, the most familiar diseases are peripheral nerve and vegetative nerve pathological changes. The representations of the peripheral nerve pathological changes of diabetes are hands and feet tingling, pain, sensory disturbance, nerve conductive velocity slower showed by electromyographic test. The representations of the vegetative nerve pathological changes of diabetes are the apparent difference of electrocardiogram showing heart rate between laying and standing, the apparent difference of heart rate between exhalation and inspiration, and sexual impotence, etc.

The diabetic pedopathy means the blood supply deficiency of feet caused by angiopathy and the hypoesthesia of feet caused by nervous lesion, together with the infection of feet. The population of amputee caused by diabetic pedoathy are 5-10 times more than those observed in non-diabetes.

It is well known that diabetes and obesity can cause cancer (National Institutes of Health, http://www.nih.gov, Title: Molecular Approaches to Diet and Pancreatic Cancer Prevention, Program Announcement Number: PA-05-040). Both cancer and angiocardiopathy are two difficulties in the field of latrology and are the top two diseases causing death.

Until now, people have recognized that the main causes of diabetes include autoimmunity system deficiencies of genetic causes, oxidative stress and environmental factors such as obesity and insufficient exercises. At present, one of the widely acknowledged theories is that the most important exopathic cause of chronic diabetic complications is the oxidative stress, accompanied by compromised immunity caused by overweight and depression, also including the actions by the genetic factors. Based on this theory, successes in therapy have been obtained. For example, a treatment using antioxidants such as Vitamin C and E, trace elements such as Selenium and Zinc, and Simvastatin, Atorvastatin, Fenofibrate, Enalapril, etc. to decrease the oxidative stress has been reported to be effective against certain chronic diabetic complications (Am J Kidney Dis. 1999, Sep., 34(3), 445-55; J Intern Med. 2005 May, 257(5), 438-45 and Eur J Pharmocol. 2004, Jun. 16, 493(1-3), 183-9 etc.) Presently, the general strategy to treat diabetes is to control the blood sugar level to prevent the acute and chronic complications, which, however, give mixed results. In addition, although insulin can maintain the circulating glucose level normal, it has no effects on internal oxidative stress, one of the most important causes to chronic diabetic complications, and low bioavailability of glucose. (Am J Clin Nutr. 2002, Apr., 75(4), 728-33). Both the type I diabetes and the serious type II diabetes need auxiliary medications in addition to insulin, and the type II diabetes at a mediate degree needs a comprehensive and effective treatment.

The clinical studies showed that the onset and development of chronic diabetic complications can be delayed, while the problem of the endocrine dysfunction left unsolved. Basically, medicines have been designed to stimulate the pancreas to secrete insulin to enhance the metabolism of the blood sugar, or to be substitutes of insulin. Such medicines can only postpone but not avoid the onset and development of chronic diabetic complications, and thus do not have the functions of preventing diabetes. Although increasing the activity of insulin is one of the most ideal means to decrease glycemia, the commercial available drugs such as Pioglitazone and Rosiglitazone can cause rises in the levels of both the total cholesterol and the low density cholesterol and liver lesions. The toxic-/side-effects of Metformin include the rise in the level of endo-homocysteine, which may indicate a increased risk of apoplexy and myocardial infarction. So, persisting with the presently available drugs may compromise the physical and the immunity status of the diabetes patients. It has been shown that, limited effectiveness can be obtained if the treatment is limited to a certain chronic diabetic complication without a panorama regimen. This may lead to acute and chronic diabetic complications and threaten the diabetes patients' life.

Various considerations should be considered to obtain an effective and comprehensive control of blood sugar and chronic complications because hyperglycemia alone, for example, has been associated to factors in many aspects and organs, such as the bioavailability of glucose, enhanced decomposition of hepatic glycogen and lowed glucose metabolism.

Providing a synergistic formulation is a very effective way of inventing new drugs in Chinese traditional medicine. Its uniqueness has been sporadically reported in studies of the western medicine. An example is a report of the result of a clinical study published on Journal of the American Dietetic Association (J Am Coll Nutr. 2004, June, 23(3), 272-9). The object of the clinical study was to evaluate the influence of a mixture of Mg+Zn, Vitamin C+Vitamin E, or their combination on the blood pressure of type II diabetes patients. The materials and method can be briefed as follows. A random, double blind clinical trial was conducted, wherein the placebo was taken as the control. 69 type II diabetes patients were divided into 4 groups randomly, and each group took the following materials for 3 months respectively: group M: daily dose of 200 mg Mg+30 mg Zn (16 patients totally); group V: daily dose of 200 mg Vitamin C+150 mg Vitamin E (18 patients totally); group MV: taking Vitamins and minerals simultaneously (17 patients totally); group P: taking placebo (18 patients totally). Both pre-trial and post-trial blood pressures were measured, and the therapeutic effectiveness was analyzed using the standard linear model. The result of the clinical trial showed that the systolic pressures, the diastolic pressures and the average blood pressures of MV group decreased the most significantly by 8 mmHg (122+/−16 vs. 130+/−19 mmHg), 6 mmHg (77+/−9 vs. 83+/−11 mmHg) and 7 mmHg (92+/−9 vs. 99+/−13 mmHg) respectively. The blood potassium levels were increased significantly and blood malondialdehyde level decreased significantly in MV group. And no apparent effectiveness was observed in the other groups. The conclusion of the clinical trial was that using the combination of the four ingredients is an effective way to decrease blood pressure in type II diabetes patients, and only using vitamins or minerals had no apparent effects. A similar clinical study using a combination of vitamins and minerals to affect blood lipid in type II diabetes patients was done, and the result was consistent with the previous one. The clinical study indicated that the average levels of both high density lipoprotein and apolipoprotein Al were effectively increased by the combination of the four ingredients, while only using vitamins or minerals had no apparent effects (Diabetes Res Clin Pract. 2004, July, 65(1), 21-8). Both of the two clinical studies proved that the advantages of an appropriate combined formulation are very significant.

The concurrent researches in medicine have shown that many materials inside the human body or functional ingredients in foods have effects on treatments of diabetes, and rationally using these materials in combination may provide a synergistic result. For example, evidences show that some compounds in the malt of barley had the similar activities of Metformin (dimethyldiguanidine) except for its side effects. Vitamin H at a level higher than the physiological level can activate the soluble guanylate cyclase, indicating that Vitamin H may function in the improvement of glucose tolerance and β cell maintenance in β cells, liver and skeletal muscles. It has been recently discovered in epidemiology that there exists an association between a proper profile of Mg and the decreased risk of diabetes and high sensitivity of insulin; the abundance of chromium picolinate can improve the sensitivity of insulin in diabetes patients, and help them to control glycemia; the combination of calcium and Vitamin can help the diabetes patients to keep the sensitivity of insulin by preventing the function of parathyroid glands from being sthenic; and etc. (Med Hypotheses. 2005, 64(1), 151-8).

### SUMMARY OF THE INVENTION

The technical problems to be resolved by the invention are to overcome the disadvantages mentioned above and to provide a synergistic formulation to prevent and/or to treat diabetes at levels of symptom and pathogenesis.

The invention provides a synergistic formulation for preventing and/or treating diabetes.

The synergistic formulation of the invention comprises Vitamin E, chromium picolinate, selenium, lutein, folic acid, Vitamin C, alpha_lipoic acid, zinc, and a pharmaceutically acceptable carrier. The said formulation may optionally comprise glutathione, lycopene, nicotinamide, L-arginine and vanadium. Further, it may optionally comprise coenzyme Q10, a vitamin complex and mineral. More further, is may optionally comprise one or more additional hypoglycemic agents such as Glimepiride, Glibenclamide, Glipizide or/and Metformin.

The capacity of said active ingredients to decrease blood sugar, to prevent and to treat the chronic diabetic complications have been reported in documents. However, when being used alone, they are not so effective as the present invention in controlling the blood sugar, preventing and treating the chronic diabetic complications.

A formulation according to the invention may comprise all of the following ingredients or several of them. The content of each ingredient is listed as below:

Generally, the content of Glimepiride is in the range of 2-10 mg, Glibenclamide is in the range of 2.5-15 mg, or Glipizide is in the range of 2.5-30 mg.

Generally, the content of Metformin is in the range of 200-1000 mg.

Generally, the content of Vitamin E is in the range of 50-3000 IU, usually 100-1200 IU, and, most preferably, 150-600 IU.

Generally, the content of chromium is in the range of 1.0-1000 μg, usually 10-500 μg, and, most preferably, 50-380 μg.

Generally, the content of selenium is in the range of 1.0-1000 μg, usually 10-680 μg, and, most preferably, 50-380 μg.

Generally, the content of lutein is in the range of 1.0-100 mg, usually 0.2-80 mg, and, most preferably, 0.5-30 mg.

Generally, the content of folic acid is in the range of 50-2000 μg, usually 100-100 μg, and, most preferably, 200-900 μg.

Generally, the content of Vitamin C is in the range of 10-2000 mg, usually 50-800 mg, and, most preferably, 100-700 mg.

Generally, the content of alpha_lipoic acid is in the range of 1.0-3000 mg, usually 5.0-1200 mg, and, most preferably, 40-700 mg. Or, the effective administration dosage of alpha_lipoic acid is 1.0-60 mg/Kg body weight, and the most preferably, 1.2-14 mg/Kg body weight.

Generally, the content of lycopene is in the range of 0.1-300 mg, usually 0.3-200 mg, and, most preferably, 0.4-100 mg.

Generally, the content of nicotinamide is in the range of 10-1800 mg.

Generally, the content of L-arginine is in the range of 100-3000 mg.

Generally, the content of zinc is in the range of 10-300 mg.

Generally, the content of vanadium is in the range of 2-300 μg.

Generally, the content of glutathione is in the range of 10-800 mg.

Generally, the content of coenzyme Q10 is in the range of 40-400 mg.

The synergistic formulation of the invention may be in either a form to provide an immediate decrease in glycemia level (immediate-decrease formulation) or a form to provide an effect of health recovery (recovery formulation).

The main active ingredients in the recovery formulation include Vitamin E, chromium, selenium, lutein, folic acid, Vitamin C, alpha_lipoic acid and zinc. It may optionally comprises glutathione, lycopene, nicotinamide, L-arginine and vanadium. Also optionally, it may further comprise coenzyme Q10, a vitamin complex and minerals.

The main active ingredients in the immediate-decease formulation are sulfonylurea-based hypoglycemic agents such as one of Glimepiride, Glibenclamide and Glipizide, Metformin, Vitamin E, chromium, selenium, lutein, folic acid, Vitamin C, alpha_lipoic acid, zinc, lycopene and/or other kinds of carotenoid, etc. The immediate-decrease formulation may also comprise a vitamin complex and minerals. A desired effect may be achieved by increasing the contents of chromium and selenium. The reported results in clinical studies (Anderson RA, et al., Diabetes, 1997, 46(11), 1786-91; and the annual Conference of NIH, Maryland, June 2006) showed that medication with abundant chromium was effective in almost very severe insulin-dependent type II diabetes. It was reported that after two weeks of the said medication, the patients no longer needed other hypoglycemic agents or insulin. The synergistic formulation of the invention is compatible with insulins, and has no cross-toxicity or side effects. For severe diabetes and type I diabetes, the said recovery formulation can be used in combination with insulin.

The function mechanism of the above mentioned effective ingredients will be detailed in the following.

Glimepiride

Glimepiride was an artificially synthesized second generation sulfonylurea hypoglycemic agent which was developed by German Hochst Mariom Roussel (HMR) in 70 s of the 20^{th} century, first launched in Swede with the Trademark as Amaryl in September 1995, and approved by FDA in 1996. Glimepiride was used to lower blood sugar through stimulating β cell of pancreas to secrete insulin, decreasing the resistance of insulin. Glimepiride was mainly used to treat type II diabetes, which can not be controlled by exercising and diet. It was the first sulfonylurea hypoglycemic agent approved by FDA that can be used with insulin simultaneously. Owing to Glimepiride's short reacting time with its receptor, Glimepiride can shorten the time of insulin secreting and has the relatively strong effect on saving insulin. Therefore, Glimepiride can get rid of the subsequent exhaustion of islet cells. The features of Glimepiride are high performance, long-acting, lower dosage (2-10 mg/d) and less side effects. It can be used with insulin or biguanide. Glimepiride has been clinically evaluated as a very good sulfonylurea hypoglycemic agent until now.

Glibenclamide

Glibenclamide was also an artificially synthesized second generation sulfonylurea hypoglycemic agent, which was mainly used to affect on β cell of islet of pancreas to increase insulin releasing and to strengthen the effect of insulin. Its effect of decreasing blood sugar was quick and long-acting. It is clinically suitable with the adult slight-medium and stable patients and can be used with insulin or biguanide.

Glipizide

Glipizide was also an artificially synthesized second generation sulfonylurea hypoglycemic agent, which was mainly used to affect on β cell of islet of pancreas. Glipizide had the effect on promoting the secretion of endogenous insulin, on inhibiting the decomposition of hepatic glycogen, and on promoting muscle to use glucose. Glipizide could enhance the effect of insulin through changing the response of target tissues toward insulin out of pancreas gland. Glipizide could strengthen the effect of insulin and could be used with insulin or biguanide.

Metformin

Metformin, such as Metformin HCl, was an artificially synthesized second generation biguanide oral hypoglycemic agent, which was mainly used to treat type II diabetes. Metformin could increase insulin-sensitivity and decrease the concentrations of fasting blood sugar and insulin. The main mechanism of it is to decrease the decomposition of hepatic glycogen by liver so that the glucose is released less by liver and is absorbed more by peripheral cells. The number of the insulin receptors can be increased by Metformin but the concentration of serum insulin will not be increased. Therefore, using Metformin can not make the patients hypoglycemosis when they have normal blood sugar. In 1994, 1996 and 1997, the researchers treated about 20 women of polycystic ovary syndrome (PCOS) with daily dose of 1500 mg Metformin HCl. 8 weeks later, the menstruation of 86.7% women were recovered to a normal level and their serum progesterone were recovered to the ovulatory period value, but their concentrations of LH and serum insulin were decreased statistically. The women can be improved of menstrual cycle and fertility after 6 months of continuous treatment. Meanwhile, the possibility of abortion could be decreased by Metformin HCl through decreasing Androgen in blood and through decreasing insulin-resistance and PAI-1. It was discovered that the hypoglycemic effect of the composition comprised of Metformin HCl and Glibenclamide was better than that of single one of them. Otherwise, the results of the clinical study showed that the concentrations of folic acid and Vitamin B12 in the blood were decreased in the patients of type II diabetes by using Metformin, and meanwhile the concentration of homocysteine was increased to a certain degree. The elevated concentration of homocysteine is an important cause of angiocardiopathy and cerebrovascular disease, such as apoplexy, etc. And the increasing of homocysteine concentration is a result of the decreasing of the concentration of folic acid and Vitamin B12 (J Intern Med. 2003 November, 254(5), 455-63). It was proved on the other hand by the result that the type II diabetes patients treated with Metformin should be supplied with folic acid and Vitamin B12.

Vitamin E

Vitamin E can apparently affect the animals' reproduction and development. Animals' reproduction function can be injured by insufficiency of Vitamin E and can be recovered by supplying it. Vitamin E is sensitive to oxygen and can be easily oxidized. Therefore, Vitamin E can preserve other substances that can be oxidized (such as unsaturated fatty acid, Vitamin A, etc.) from being oxidized in vivo, and Vitamin E can prevent excessive Vitamin A by promoting the absorption, usage, and reservation of Vitamin A by liver.

Free radical is a kind of active group existing in various kinds of chemical reaction, which has important function on human normal physiological metabolism. The free radical-chain reaction aroused by excessive free radicals can induce lipid peroxidating of the unsaturated fatty acid on the cell membrane. The macromolecule protein and nuclear acid in the cells and on the membrane are damaged by the newly produced large amount of lipid peroxide so that the organism is hurt. When the free radicals enter into lipid phase and initiate the chain reaction, Vitamin E will scavenge the free radicals. Vitamin E has the high efficiency of resisting the lipid peroxidation by free radicals. Vitamin E can decrease the level of profibrinolytic activating factor inhibitor-1 and that of the P-selectases, which are the markers of thrombosis of diabetes patients or health people. So, Vitamin E can be used as an additive medicine for treating thrombosis of atherosclerosis (Diabetes Care. 2002, March, 25(3), 524-9). It was discovered in the researches that the insufficiency of Vitamin E would have the effect on the immune function of both human and animal. The insufficiency of Vitamin E not only decreases the humoral immunity but also has great effect on cellular immunity.

The results of the clinical study, which was published on Sep. 24, 2004 by the scientists of New Zealand, showed that Vitamin E could improve the activity of insulin and could strengthen the function of liver.

The researches in 80 s of the 20^{th}. century also showed that Vitamin E was one of the important protective factors of the growth of liver cells. It was discovered by the researches that one of the dying paths of liver cells was the exhaustion of Vitamin E in liver cells. Vitamin E has the protective function on various kinds of the acute hepatic injuries and has delaying function on the chronic hepatic fibrosis. The levels of Vitamin A and E in the bodies of all diabetes patients are much less than that in the bodies of health people (Gen Physiol Biophys. 2003, Mar., 22(1), 15-27), and the diabetes patients must be supplied Vitamin A and E. The immunity can be improved, the chronic complications of diabetes such as angiocardiopathy, cerebrovascular disease, diabetic renopathy and diabetic hepatopathy can be prevented and treated if the diabetes patients take abundant Vitamin E. Diabetic neuropathy can affect each part of the nervous system of the human body, such as to destroy the brain and the spinal cord, and can arouse dementia.

The specific article of NIH indicated that the insufficiency of Vitamin E was one of the important causes of destroying the nervous system of diabetes patients. http://www.nlm.nih.gov/medlineplus/ency/article/001161.htm. In addition, the discussion of a specific article of NIH published in February 2004 indicated that the two individual clinical studies showed that Vitamin E could prolong the life of dement and promote their daily activity amount.

Chromium

Chromium is one of the basic elements for human metabolism. It is proven that chromium can help the body decreasing fat and increasing muscle simultaneously, and can help the body keeping a normal level of blood sugar. The best chromium product is Chromium Picolinate. In 50 s of the 20^{th}. century, the white mice were fed with the beer yeast to improve their abnormal sugar metabolism and the treatment effect of the beer yeast was observed in the field of medicine. These mice were metabolic disturbance because of being fed with the feedstuff without chromium. At present, chromium is considered as a kind of necessary microelement adjusting the sugar metabolism. Oligopeptide formed via chromium oligermization participates in stimulating and conducting insulin after complex with insulin receptor. The diabetes patients' capacities of both secreting insulin or/and the function of the secreted insulin are deficient, especially that of type II diabetes patients. (http://www.clevelandclinic.org/heartcenter/pub/news/achive/2003/chromium4_{—}02.asp) The report of US Cleveland Heart center on Apr. 3, 2003 indicated that chromium could improve the insulin function and could decrease some risk factors of insulin-resistance diseases, such as obesity, angiocardiopathy, type II diabetes, polycystic ovary syndrome (PCOS), and atypical depression, etc. Therefore, supplying suitable amount of microelement chromium could improve diabetes patients' ability of sugar metabolism, could strengthen type II diabetes patients' glucose using (orv Hetil. 2003, 144(42), 2073-6), so as to control the diabetes patients' blood sugar and meanwhile achieve the aim of preventing and delaying the occurrence and development of the chronic complications of diabetes. Many clinical studies showed that the severe diabetes patients who need to use insulin did not need insulin anymore after they had taken the abundant amount of microelement chromium for two weeks (Diabetes, 1997, 46(11), 1786-91 and J Trace Elem Exp Med, 1995, 8, 183-90, etc.). The therapeutic effect of the formulation comprising chromium and other ingredients was better than that of using the ingredients alone. For example, it was discovered by the very early research that using chromium and Vitamin B3 together could decrease the concentration of fasting blood sugar and could increase the glucose tolerance (Metabolism 1987; 36, 896-99). The mechanism of chromium controlling diabetes patients' blood sugar is similar to that of Metformin, but chromium does not have the toxicity/side effects of Metformin's.

Selenium

Selenium was discovered in the recent 20 years to be a substance having great influence on human's health condition. It was discovered by the animal test that the activity of the nature killer cells in the mice's body could be strengthened when the mice drank the water with the additive selenium. It was proved in vitro that selenium could cause the apoptosis of cancer cells. It was known that selenium could improve immunity and had the effect of preventing cancer.

Vitamin E and selenium are the substances with synergistic action and have stronger effectiveness when taken together and have fairly weaker effectiveness when taken separately. Both Vitamin E and selenium are the anti-oxidants, which can prevent and delay the phenomena of aging and sclerosis of human tissues caused by oxidation. Selenium can keep the activity of human tissues and alleviate suffering of burns and menolipsis. It was reported by CNN98/8/22 that selenium, which was discovered having the capacity of anti-cancer and anti-oxidation, could decrease ½-⅓ of the occurrence of prostate cancer. More than 40 thousand men die of prostate cancer every year in US, which is becoming the No. 1 killer. It was discovered by the researches done comparing the American men with high selenium diet and those with low selenium diet by National Cancer Institute. It was shown by the research results that not only diabetes and its chronic complications could be treated by selenium (Biomedicine & Pharmacotherapy, 1998, Vol 52, 89-95 and Journal of Endocrinology, 2005, 184, 455-465), but also be prevented by selenium, and the blood sugar lowering effect of selenium is similar to insulin. The insulin mimic effects of selenium included glycolysis, phosphopentose pathway and the synthesis of fatty acid (Biomed. Pharmacother. 1998, 52, 89-95). Selenium also can decrease oxidative stress, which means to decrease the injury on cells aroused by the unbalance between oxidant and antioxidant. Thus it can be seen that supplying selenium is very important to diabetes patients. Selenium can improve the immunity of the patients, especially for male diabetes patients.

Lutein

Lutein is the most important nutritious ingredients for human retina. The macula of retina (vision center) and the crystal body contain large amount of lutein. Lutein can not be synthesized in vivo and must be absorbed from the outside.

It was shown by the research results of Association for Research in Vision and Opthalmology (ARVO) that lutein could improve the vision damaged by dry aging macular degeneration (AMD). AMD is the main disease causing patients blind in the western countries. About 30 million people are suffered from the disease and it is estimated that the population will be increased 2 times in 2030. It was discovered by the researches that taking the purified lutein nutrition supplement or taking the mixture supplement of lutein with other anti-oxidants (such as Vit A, Vit C, Vit E or β-carotene) could apparently improve the AMD syndromes. It was shown by a research supported by State Department in Vision that taking the substances full of carotenoid including lutein could decrease the risk of having AMD (JAMA, 1994, 272, 1413-1420).

Lutein was proven to be an important natural antioxidant. Research of Hawaii Cancer Center discovered that lutein is one of the most effective ingredients inhibiting lipid oxidation and the oxidation happening in blood and eyes. Lutein could resist the damage caused by these free radicals, especially in the field of the retina and the crystal body.

It was shown by a report from US sent by Retinal International to its subsidiaries that lutein is useful for improving the vision of the patients with pigmentary degeneration of retina and other degenerations of retina. It was the first news about the effectiveness of lutein from an international body of retinal degeneration patients. A report captioned of “Lutein Supplements May Improve Vision” was published by School of Medicine of Johns Hopkins University. The participants of the research took lutein supplement every day for 6 months or more. The vision of the 12 patients was apparently improved among the 16 patients with retinal degeneration in the research. Diabetic fundus is one of the most popular chronic complications of diabetes among the diabetes patients. It was shown that supplying abundant amount of lutein for diabetes patients could improve the anti-oxidation ability of their eyes, and could prevent and treat diabetic fundus.

Folic Acid

Folic acid naturally exists in the animal's liver and kidney. Folic acid is in Vitamin B family consisted of pteridine, para-aminobenzoic acid and glutamic acid, which is a necessary substance for cell growth and reproduction and is water soluble vitamin assisting the maturation of erythrocyte. It was shown by the animal experiments that 71%-88% babies were aplasia if their mothers had diabetes or the embryo was put in the environment of the similar glucose concentration. The probability of babies' aplasia was decreased to 3% or 5% if the pregnant animals were fed with folic acid or the embryos were put in the environment of the similar glucose concentration with folic acid (Diabetes, 2005, 54, 546-553). The manufacturers of wheat flour were forced by FDA on Apr. 27, 2004 to add folic acid into the processed white flour to prevent heart disease and apoplexy, because the folic acid would be destroyed during the processing procedures. It was shown by the new researches that folic acid not only could prevent the congenital defect of the neonate, but also could prevent stroke.

An article published on Journal of the American Medical Association (1998, Vol 279, 359-364) was about a 14 year-long-term research accomplished by School of Public Health of Harvard University, which was a rare large-scale clinical research with 80,000 samples. The groups of taking large amount of folic acid and Vitamin B6 were compared with those taking small ones by the research. The groups taking large amount had daily dose of 700 mg folic acid and 4.5 mg Vitamin B6. The groups taking small amount had daily dose of US RDA or less. It was discovered that occurrence of heart disease decreased 45% in the groups taking large amount.

In addition, it was discovered that folic acid and Vitamin B12 could treat and prevent heart disease (Folic acid and Vitamin B12 for heart disease treatment, September 2001). The research was carried out by University of California (San Francisco) and it was shown by the result that heart disease was effectively prevented by folic acid and Vitamin B12, which was published on Journal of American Medical Association. The level of homocysteine is an important index for the risk of heart disease. If the level is higher than the normal one, the risk of stroke, heart attack and the risk of death aroused by them are much more higher. It was shown by the results of the random clinical study done by University of California (San Francisco) that folic acid could decrease the level of homocysteine by 25%. It was shown that the effectiveness was higher if Vitamin B12 was added, which could decrease the level of homocysteine by 7% more. Supplying the abundant amount of folic acid could improve the life-force of the cells of the patients' body and could prevent and treat diabetic microangiopathy well.

Glutathione

Glutathione (L-glutamic acid-L-cysteine-glycine) exists in all animal cells. It stays in reducing form of thio-alcohol (GSH) under the normal environment and is a main nonprotein sulfhydryl compound in the cells. It has direct or indirect function on many life activities, including regulating and controlling gene expression, regulating the activities and metabolism of enzymes, protecting cells, amino acids transporting, and regulating immunity function, etc. Oxidative stress or the attacking by the electrophilic compounds lowers the content of GSH in the cells, or makes GSH changed to oxidized dithion form (GSSG), and GSSG can be changed to GSH through glutathione reductase having NADPH as the coenzyme. Glutathione was used clinically for detoxification, antianaphylaxis, and to treat cataract, etc. It was discovered recently that glutathione had the function of anti-oxidation and regulating body's thio-balance, it also had the function of neurotransmitters, or neuroregulation in nervous system. Glutathione was clinically used as the medicine for detoxification and anti-oxidation. Cai Weiping et al. used glutathione to treat various acute medicamentous kidney lesion and being controlled with the traditional composition of amino acids and Jinshuibao capsule (Cai Weiping, Liao Lutan, Glutathione treating acute medicamentous kidney lesion (J), Chinese Journal of New Drugs and Clinical Remedies, 2000, 19(4): 291). It was shown by the result that the total effectiveness of glutathione and control was 92% and 64% respectively (P<0.05), the total effectiveness on blood urine was 89% and 85% respectively (P<0.01), and the total effectiveness on proteinuria was 57% and 50% respectively (P<0.05). It was shown that glutathione was useful for kidney function recovering and the recovery duration shortened. It was shown that glutathione had the apparent effect on hematuria and proteinuria which were no harm on kidney function. It was shown that the side effects of glutathione were little and not serious. When the content of selenium and alpha_lipoic acid in the composition preparation of the invention was as the amount listed in the following tables, no glutathione was needed.

Vitamin C

Vitamin C is a necessary vitamin for human and is considered as an important role in cell breath redox reaction. Vitamin C is needed for forming and maintaining intercellular substances and collagen, for steroid synthesis, for transforming folic acid and for the metabolism of tyrosine. Vitamin C is a necessary antioxidant for tissues growth and for amending health gum. Vitamin C has various functions of improving blood circulation, eliminating fatigue, improving leukocyte function, strengthening immunity, protecting nervous system, improving metabolism, preventing scurvy, preventing fracture, etc. Vitamin C also can decrease cholesterol and hypertension and can prevent atherosclerosis.

It was discovered that diabetic heart disease had the relationship with low of Vitamin C in vivo (Journal of Diabetes and Its Complications 1998; 12: 259-263). It was discovered in a 16-year clinical study accomplished by School of Public Health of Harvard University, which including 85 thousand women, that the diabetes patients with daily dose of 400 mg or more Vitamin C had been decreased the risk of having fatal and nonfatal coronary heart disease greatly (J Am Coll Cardiol. 2003, 42(2), 246-252). The diabetes patients usually have nephropathy, the most serious disease caused by nephropathy is heart disease. It was discovered by the researchers from 37 diabetes cases that the concentration of Vitamin C in vivo of the patients having diabetic nephropathy were lower than that of those not developing nephropathy. It was discovered that their clearance rate of Vitamin C by kidney were quicker. It might be due to easily running off Vitamin C from kidney by nephropathy and therefore the concentration of Vitamin C in vivo decreased. It was inferred audaciously by the researchers that insufficiency of the protection of the vitamin anti-oxidant was the main cause for increased risk of heart disease of the patients with nephropathy. So, it was recommended to comprising more Vitamin C in the composition preparation of the invention to supply the lost of quickly running off Vitamin C from kidney. Therefore, it was possible to promote the capacity of anti-oxidation of the in vivo tissues of diabetes patients so as to prevent and treat diabetic nephropathy well (Diabet Med. 2001, 18(9), 756-60) and to prevent and treat angiocardiopathy, cerebrovascular diseases. It was also discovered that the diabetes patients could be helped controlling blood sugar by Vitamin C stimulating the insulin mechanism of the patients (In Vivo. 1993, 7(6A), 535-42).

Alpha_Lipoic Acid

Alpha_lipoic acid is a natural antioxidant in human body produced by mitochondrion. Alpha_lipoic acid increases both the intracellular and extracellular concentration of water soluble Vitamin C and liposoluble Vitamin E simultaneously. Alpha_lipoic acid can regenerate Vitamin C and E by its redox feature. Alpha_lipoic acid can be a “substitute” when other antioxidants are deficient. At present, alpha_lipoic acid is the most effective natural antioxidant among those known by people.

Alpha_lipoic acid can be used to treat hepatonecrosis, hepatitis B and C. An American doctor had treated 3 patients with hepatonecrosis produced by poisonous mushroom with alpha_lipoic acid. As a result, the illness of the 3 patients had been controlled within a short period and their liver function had been recovered. Alpha_lipoic acid can combine with the toxin in the liver and decompose the toxin so that it can relieve hepatitis syndrome and recover the liver function. Alpha_lipoic acid can destroy various kinds of different free radicals and regenerate other antioxidants to destroy the free radicals. The defense system of anti-oxidation of patients carrying HIV is usually weak. Owing to insufficiency of antioxidant, the reproduction of virus can not been prevented when the virus is stimulated by oxidant. Alpha₁₃ lipoic acid can stimulate the concentrations of Vitamin C, total glutathione and total sulfide in blood to increase and can improve the ratio of T4/T8 lymphocyte to decrease the injury caused by free radicals on patients.

The researches of its effects on treating AIDS, Parkinson's disease, Alzheimer's disease and other diseases are going on in US. In Mayo, 120 patients were randomly divided into two groups and were accepted intravenous injection of 600 mg alpha_lipoic acid and placebo respectively for 5 times. It was discovered only after two weeks that the symptoms of the patients using alpha_lipoic acid group were improved apparently, such as the feeling of pain was decreased 6 points, while that of control group was decreased 2 points. It was discovered that the effects of alpha_lipoic acid were not only relieving pain and other symptoms, but also improving the metabolism condition of nerve or blood vessel.

In addition, the difference between alpha_lipoic acid and Vitamin E is its solubility both in water and lipid, which makes it being able to enter into all cells in vivo. It was shown by the research that alpha_lipoic acid had strong effect on inhibiting the oxidation of protein, which has the close relationship with aging and Alzheimer's disease. Alpha_lipoic acid, the antioxidant, was useful for preventing heart disease and stroke. Alpha_lipoic acid could increase the effect of insulin in the circulation of diabetes patients and decrease blood sugar.

It was discovered by the recent researches that alpha_lipoic acid also had other beneficial effects on human body, such as auxiliary treating of type II diabetes by improving the function of islets of pancreas and the glucose metabolism. Supplying alpha_lipoic acid could improve the function of islets of pancreas of type II diabetes patients, increasing insulin sensitivity and strengthening glucose metabolism (Free Radic Biol Med. 1999 Aug., 27(3-4), 309-14). It could increase combustion using of glucose to decrease blood sugar (Drug Dev Ind Pharm. 2004 Jan., 30(1), 35-42). Meanwhile, it could improve the blood sugar controlling of diabetes patients and help them to decrease the dosage of insulin or hypoglycemic agents. One of the complications of diabetes is diabetic neuropathy, which could be relieved by alpha_lipoic acid and be prevented for the patients without neuropathy (Diabet Med. 2004 Feb., 21(2), 114-21). In addition, glutathione is a kind of important antioxidant consisted of three amino acids which can prevent cataract. Alpha_lipoic acid has several kinds of biochemical functions as that of glutathione, such as maintaining the concentration of Vitamin C in blood, ensuring the recycle of Vitamin E, and alpha_lipoic acid can also prevent cataract.

Lycopene

Lycopene is one kind of carotenoid. Once lycopene contacted with oxygen, it will be converted to lycopene epoxide which is a kind of antioxidant being increased 40% of weight. Lycopene is the strongest antioxidant for eliminating liposoluble free radicals in vivo (Vitamin C can eliminate the water soluble free radicals). Lycopene can inhibit formation of lipid peroxide and can prevent adult diseases, including preventing angiocardiopathy and prostatic carcinoma or malignant tumor of digestive tract, inhibiting carcinoma of large intestine, carcinoma of colon and carcinoma of urinary bladder, preventing hypertension, decreasing blood lipid, resisting cell damage, protecting DNA, proteins, fat and lipid, etc. of cells in vivo. It was indicated by the epidemiological primary survey that people who have lower lycopene concentration would have high occurrence rate of cancer of pancreas and carcinoma of urinary bladder. High concentration of lycopene are in testicle, adrenal gland and prostate. Lycopene regulates cancer inhibition gene, decreases the occurrence rate of cancer and it was shown in vitro that lycopene could inhibit the proliferation of cancer cells. Using lycopene alone could not inhibit the proliferation of prostatic carcinoma strongly, but it could inhibit the proliferation of prostatic carcinoma effectively combining with α-tocopherol (Vitamin E). It was indicated by the American clinical study that the cancer cells proliferation were stopped in 44% of the patients. The size of the tumor were not over 4 dm³ in 85% of the patients of prostatic carcinoma when the extraction of lycopene was taken by the patients 3 weeks before the operation. And the size of the tumor were not over 4 dm³ in 55% of the patients of prostatic carcinoma when they had not taken the extraction of lycopene. Besides the above, lycopene was also effective on cancer of lung, carcinoma of colon and cancer of pancreas. Little dosage of lycopene could prevent carcinoma of colon. It was shown by the clinical studies of European multiple medical centers that lycopene could decrease the risk of myocardiac infarction. It was shown by a clinical study of 400 subjects that lycopene could prevent stomach wound from transforming precancerous lesion. It was shown by the test of multiple medical centers of 5 cities in Japan that the concentration of lycopene in blood could be used to forecast carcinoma of stomach. It meant that lycopene could promote the capacity of anti-oxidation of diabetes patients and could prevent the patients from producing various carcinogenesis because of their low immunity.

Other Effective Ingredients Including Vitamin B, L-Arginine, Microelement Zinc, Vanadium

L-arginine was mainly used to treat diabetic microangiopathy and to promote insulin sensitivity. Vitamin B family, such as nicotinic acid, was used to treat the chronic complications of diabetes caused by abnormity of lipid and to promote the immunity of diabetes patients. Microelements, such as zinc and vanadium, were used to treat oxidative stress of diabetes, to strengthen blood sugar metabolism and to promote the immunity of diabetes patients. The basic level of C-polypeptide was kept unchanged for two years by using the combination of nicotinamide and Vitamin E or by using nicotinamide only together with intensive insulin treatment. It was the newest discovery that was very important for the prepuberal type I diabetes patients (Eur J Endocrinol. 2004, May, 150(5), 719-24). L-arginine was praised as “a magic molecule” because of its strong recovering ability, which was discovered as the most abundant natural product. It was signed out by Nobel Prize that nitric oxide (NO) is necessary for human life, and L-arginine is the main source of NO in human body. Taking L-arginine orally could improve the expanding function of endothelial cells of type II diabetes patients (Vasc Med. 2003, 8(3), 169-75).

Vitamin Complexes and Minerals are Well Known Substances for Keeping Health in the Art.

In addition to the above mentioned active ingredients in the invention, it also comprised the pharmaceutical acceptable carriers.

Basically speaking, almost all excipients used in vitamin complexes could be used as excipients in the invention. For example, corn starch could be used as filler and disintegrant, cellulose gel could be used as plasticizer and adhesives, gelatin could be used as emulsifying agents, adhesives and disintegrant, glycerin could be used as sweetener, hydroxypropyl cellulose could be used as water dispersion material, magnesium/zinc stearate could be used as lubricant, croscarmellose sodium could be used as adhesive and disintegrant, lactose could be used as filler and adhesive, acacia could be used as emulsifying agent and adhesive, stearic acid could be used as emulsifying agent and adhesive, hydroxypropyl methylcellulose could be used as adhesive and disintegrant, sugar could be used as corrigent, polyethylene glycol could be used as filler, emulsifying agent and disintegrant, modified food starch could be used as filler and disintegrant, soybean oil and lecithin, etc. could be used as liposoluble additive materials, and titanium dioxide could be used as coloring agent, etc.

The composition preparation of the invention can be tablet, pill, capsule, coated tablet, aerosol or liquid medicament. The liquid medicament can be alcohol solution or water solution, or suspension and emulsion, etc. All active ingredients will be chosen to use their salt under the circumstance of selecting the liquid medicaments. For a solid medicament, it can be a final medicament with all active ingredients in it, or it can be a final medicament comprising various individual medicaments involving the individual active ingredient.

Manners of Administration:

The formulation of the present invention may be orally administrated once a day in form of, for example, tablet, pill, capsule and coated tablet. The therapeutic effective amount for a given condition depends on various factors including, for example, body weight, age, gender, symptoms, severity of the disease, path of administration, etc.

Many known techniques can be used to produce a formulation according to the invention in solid form. If all active ingredients are to be contained in a single final formulation, the active ingredients can be first mixed with one or two excipients by wet-mixing or dry-mixing, and then, additional active ingredients and excipients may be added and mixed. The mixing process may be a process of granulating, slugging, blending and the like. The homogenized mixture can then be compressed into tablets or be rolled into pills.

When plant oil such as palm oil, coconut oil, palm-nut oil, soybean oil, safflower oil, canola oil, grape-kernel oil, cotton seed oil and the like is used as lipid or a lipo-soluble adjuvant, soft capsules can be prepared.

Many known techniques can be used to produce the soft capsule of the invention. For example, the active ingredients, the lipids or the lipo-soluble adjuvant may first be mixed with one or two excipients, and then, additional active ingredients and excipients may be added, and the mixture was homogenized to be prepared into soft capsules.

The present invention supply minerals, vitamins and other essential non-nutrients which are usually in scanty in diabetes patients, and thereby helps in maintaining the homeostasis, decreasing oxidative stress in vivo, enhancing immunity, repairing and protecting major organs. Further, the invention provides effective means to prevent and/or treat the chronic diabetic complications and to prevent the acute diabetic complications. Insulin and conventional synthetic small molecules are not expected to be able to adjust the endocrine homeostasis. The invention helps the diabetes patients to maintain a high-quality life and prevents the possible acute/chronic diabetic complications.

The combined formulation of the invention can be self-administrated by the patient at home and can be taken for a long course, without the need for the doctors' monitoring. This helps to decrease the medical cost and decrease the risk of latrogenic infections due to the patients' compromised immunity. The formulation of the invention has good tolerance and little drug-resistance.

In addition, The defensive anti-oxidation system in HIV patients are usually weak. Because of the deficiency of antioxidants, the viral proliferation in response to oxidant stimulation can not be controlled. Vitamin E, lutein, folic acid, Vitamin C, alpha_lipoic acid, lycopene, minerals, zinc, coenzyme Q10, etc. are effective antioxidants that can be found in vivo. And meanwhile, alpha_lipoic acid acid and Vitamin E have been proved to be effective in inhibiting retroviruses. The combined formulation of the invention can also be used in therapy or auxiliary therapy of AIDs to improve the patients' life quality. There are no undesired interactions among the active ingredients in the formulation of the invention.

### EMBODIMENTS OF THE INVENTION

The following examples are only provided to illustrate the invention, with no means to have the invention limited thereby. All experiments were conducted in standard ways or in accordance to the manufacturers' instructions, unless otherwise specified.

### Example 1

I. The active ingredients were slightly different between the male and the female, and were shown in Table 1.

TABLE 1

Amounts

| active Ingredients | Female | | Male | |
|---|---|---|---|---|
| Vitamin A | 5000 | I.U. | 5000 | I.U. |
| Vitamin C | 500 | mg | 580 | mg |
| Vitamin D | 400 | I.U. | 400 | I.U. |
| Vitamin E | 440 | I.U. | 460 | I.U. |
| Vitamin K | 20 | μg | 20 | μg |
| Thiamin | 1.5 | mg | 1.5 | mg |
| Riboflavin | 1.7 | mg | 1.7 | mg |
| Niacin | 20 | mg | 20 | mg |
| Vitamin B6 | 3 | mg | 3 | mg |
| Vitamin B12 | 25 | μg | 25 | μg |
| Biotin | 20 | μg | 30 | μg |
| Pantothenic Acid | 10 | mg | 10 | mg |
| Folic Acid | 800 | μg | 800 | μg |
| Calcium | 400 | mg | 200 | mg |
| Phosphorus | 48 | mg | 48 | mg |
| Iodine | 150 | μg | 150 | μg |
| Magnesium | 100 | mg | 120 | mg |
| Zinc | 15 | mg | 15 | mg |
| Selenium | 200 | μg | 220 | μg |
| Copper | 2 | mg | 2 | mg |
| Manganese | 2 | mg | 2 | mg |
| Chromium | 350 | μg | 350 | μg |
| Molybdenum | 75 | μg | 75 | μg |
| Chloride | 70 | mg | 70 | mg |
| Potassium | 80 | mg | 120 | mg |
| Boron | 150 | μg | 150 | μg |
| Nickel | 5 | μg | 5 | μg |
| Iron | 18 | mg | | |
| Silicon | 2 | mg | 2 | mg |
| Vanadium | 10 | μg | 10 | μg |
| Lutein | 2400 | μg | 2600 | μg |
| Alpha Lipoic Acid | 60 | mg | 60 | mg |
| Lycopene | 400 | μg | 800 | μg |
| L-arginine | 100 | mg | 100 | mg |

I.U. means International Unit, which is the quantitative unit of Vitamin A and D.

II. The general method of preparing solid dosage form includes the following steps:

1. Mixing the active ingredients and excipients, and homogenizing the obtained mixture;

2. Compressing the homogeneous product of step 1 into small granules;

3. Mixing a lubricant such as magnesium stearate with the small granules obtained in step 2 for several minutes;

4. Compressing the mixture of step 3 into the tablets or other solid dosage forms;

5. Spraying onto the tablets obtained in step 4 a coating solution as desired.

In the case of soft capsules, the homogeneous product of step 1 may be formed into soft capsules directly.

### Example 2

Sample 2 was prepared according to the procedure of Example 1 with the modifications as shown in Table 2.

TABLE 2

Amounts

| | | |
|---|---|---|
| Active Ingredients | Female | Male |

| | | | | |
|---|---|---|---|---|
| Vitamin A | 5000 | I.U. | 5000 | I.U. |
| Vitamin C | 500 | mg | 580 | mg |
| Vitamin D | 400 | I.U. | 400 | I.U. |
| Vitamin E | 440 | I.U. | 460 | I.U. |
| Vitamin K | 20 | μg | 20 | μg |
| Thiamin | 1.5 | mg | 1.5 | mg |
| Riboflavin | 1.7 | mg | 1.7 | mg |
| Niacin | 20 | mg | 20 | mg |
| Vitamin B6 | 3 | mg | 3 | mg |
| Vitamin B12 | 25 | μg | 25 | μg |
| Biotin | 20 | μg | 30 | μg |
| Pantothenic Acid | 10 | mg | 10 | mg |
| Folic Acid | 800 | μg | 800 | μg |
| Calcium | 400 | mg | 200 | mg |
| Phosphorus | 48 | mg | 48 | mg |
| Iodine | 150 | μg | 150 | μg |
| Magnesium | 100 | mg | 120 | mg |
| Zinc | 15 | mg | 15 | mg |
| Selenium | 200 | μg | 220 | μg |
| Copper | 2 | mg | 2 | mg |
| Manganese | 2 | mg | 2 | mg |
| Chromium | 350 | μg | 350 | μg |
| Molybdenum | 75 | μg | 75 | μg |
| Chloride | 70 | mg | 70 | mg |
| Potassium | 80 | mg | 120 | mg |
| Boron | 150 | μg | 150 | μg |
| Nickel | 5 | μg | 5 | μg |
| Iron | 18 | mg | | |
| Silicon | 2 | mg | 2 | mg |
| Vanadium | 10 | μg | 10 | μg |
| Lutein | 2400 | μg | 2600 | μg |
| Alpha Lipoic Acid | 60 | mg | 60 | mg |
| Lycopene | 400 | μg | 800 | μg |
| Coenzyme Q10 | 50 | mg | 60 | mg |

### Example 3

Sample 3 was prepared according to the procedure of Example 1 with the modifications as shown in Table 3. This formulation was designed for severe diabetes patients.

TABLE 3

Amounts

| Active Ingredients | Female | | Male | |
|---|---|---|---|---|
| Vitamin A | 3800 | I.U. | 4000 | I.U. |
| Vitamin C | 580 | mg | 620 | mg |
| Vitamin D | 400 | I.U. | 400 | I.U. |
| Vitamin E | 460 | I.U. | 490 | I.U. |
| Vitamin K | 20 | μg | 20 | μg |
| Thiamin | 1.5 | mg | 1.5 | mg |
| Riboflavin | 1.7 | mg | 1.7 | mg |
| Niacin | 20 | mg | 20 | mg |
| Vitamin B6 | 4 | mg | 4 | mg |
| Vitamin B12 | 25 | μg | 25 | μg |
| Biotin | 20 | μg | 30 | μg |
| Pantothenic Acid | 10 | mg | 10 | mg |
| Folic Acid | 840 | μg | 870 | μg |
| Calcium | 400 | mg | 200 | mg |
| Phosphorus | 48 | mg | 48 | mg |
| Iodine | 150 | μg | 150 | μg |
| Magnesium | 100 | mg | 120 | mg |
| Zinc | 15 | mg | 15 | mg |
| Selenium | 320 | μg | 380 | μg |
| Copper | 2 | mg | 2 | mg |
| Manganese | 2 | mg | 2 | mg |
| Chromium | 460 | μg | 480 | μg |
| Molybdenum | 75 | μg | 75 | μg |
| Chloride | 70 | mg | 70 | mg |
| Potassium | 80 | mg | 120 | mg |
| Boron | 150 | μg | 150 | μg |
| Nickel | 5 | μg | 5 | μg |
| Iron | 18 | mg | | |
| Silicon | 2 | mg | 2 | mg |
| Vanadium | 20 | μg | 20 | μg |
| Lutein | 20 | mg | 30 | mg |
| Alpha Lipoic Acid | 160 | mg | 200 | mg |
| Lycopene | 600 | μg | 900 | μg |

### Example 4

### Formulation for Lowering Blood Sugar Quickly

TABLE 4

Amounts

| Active Ingredients | Female | | Male | |
|---|---|---|---|---|
| Glimepiride | 5 | mg | 5 | mg |
| Vitamin C | 500 | mg | 580 | mg |
| Vitamin D | 400 | I.U. | 400 | I.U. |
| Vitamin E | 440 | I.U. | 460 | I.U. |
| Vitamin K | 20 | μg | 20 | μg |
| Thiamin | 1.5 | mg | 1.5 | mg |
| Riboflavin | 1.7 | mg | 1.7 | mg |
| Niacin | 20 | mg | 20 | mg |
| Vitamin B6 | 3 | mg | 3 | mg |
| Vitamin B12 | 25 | μg | 25 | μg |
| Biotin | 20 | μg | 30 | μg |
| Pantothenic Acid | 10 | mg | 10 | mg |
| Folic Acid | 800 | μg | 800 | μg |
| Calcium | 400 | mg | 200 | mg |
| Phosphorus | 48 | mg | 48 | mg |
| Iodine | 150 | μg | 150 | μg |
| Diformin HCl | 500 | mg | 500 | mg |
| Zinc | 15 | mg | 15 | mg |
| Copper | 2 | mg | 2 | mg |
| Manganese | 2 | mg | 2 | mg |
| Selenium | 200 | μg | 250 | μg |
| Molybdenum | 75 | μg | 75 | μg |
| Chloride | 70 | mg | 70 | mg |
| Potassium | 80 | mg | 120 | mg |
| Boron | 150 | μg | 150 | μg |
| Nickel | 5 | μg | 5 | μg |
| Silicon | 2 | mg | 2 | mg |
| Vanadium | 10 | μg | 10 | μg |
| Lutein | 2400 | μg | 2600 | μg |
| Alpha Lipoic Acid | 60 | mg | 60 | mg |

### Example 5

### Formulation for Lowering Blood Sugar Quickly

TABLE 5

Amounts

| Active Ingredients | Female | | Male | |
|---|---|---|---|---|
| Glibenclamide | 6 | mg | 6 | mg |
| Vitamin C | 500 | mg | 580 | mg |
| Vitamin D | 400 | I.U. | 400 | I.U. |
| Vitamin E | 440 | I.U. | 460 | I.U. |
| Vitamin K | 20 | μg | 20 | μg |
| Thiamin | 1.5 | mg | 1.5 | mg |
| Riboflavin | 1.7 | mg | 1.7 | mg |
| Niacin | 20 | mg | 20 | mg |
| Vitamin B6 | 3 | mg | 3 | mg |
| Vitamin B12 | 25 | μg | 25 | μg |
| Biotin | 20 | μg | 30 | μg |
| Pantothenic Acid | 10 | mg | 10 | mg |
| Folic Acid | 800 | μg | 800 | μg |
| Calcium | 400 | mg | 200 | mg |
| Phosphorus | 48 | mg | 48 | mg |
| Iodine | 150 | μg | 150 | μg |
| Diformin HCl | 500 | mg | 500 | mg |
| Zinc | 15 | mg | 15 | mg |
| Copper | 2 | mg | 2 | mg |
| Manganese | 2 | mg | 2 | mg |
| Selenium | 200 | μg | 250 | μg |
| Molybdenum | 75 | μg | 75 | μg |
| Chloride | 70 | mg | 70 | mg |
| Potassium | 80 | mg | 120 | mg |
| Boron | 150 | μg | 150 | μg |
| Nickel | 5 | μg | 5 | μg |
| Silicon | 2 | mg | 2 | mg |
| Vanadium | 10 | μg | 10 | μg |
| Lutein | 2400 | μg | 2600 | μg |
| Alpha Lipoic Acid | 60 | mg | 60 | mg |

### Example 6

### Formulation for Lowering Blood Sugar Quickly

TABLE 6

Amounts

| Effective Ingredients | Female | | Male | |
|---|---|---|---|---|
| Glipizide | 5 | mg | 5 | mg |
| Vitamin C | 500 | mg | 580 | mg |
| Vitamin D | 400 | I.U. | 400 | I.U. |
| Vitamin E | 440 | I.U. | 460 | I.U. |
| Vitamin K | 20 | μg | 20 | μg |
| Thiamin | 1.5 | mg | 1.5 | mg |
| Riboflavin | 1.7 | mg | 1.7 | mg |
| Niacin | 20 | mg | 20 | mg |
| Vitamin B6 | 3 | mg | 3 | mg |
| Vitamin B12 | 25 | μg | 25 | μg |
| Biotin | 20 | μg | 30 | μg |
| Pantothenic Acid | 10 | mg | 10 | mg |
| Folic Acid | 800 | μg | 800 | μg |
| Calcium | 400 | mg | 200 | mg |
| Phosphorus | 48 | mg | 48 | mg |
| Iodine | 150 | μg | 150 | μg |
| Diformin HCl | 500 | mg | 500 | mg |
| Zinc | 15 | mg | 15 | mg |
| Copper | 2 | mg | 2 | mg |
| Manganese | 2 | mg | 2 | mg |
| Selenium | 200 | μg | 250 | μg |
| Molybdenum | 75 | μg | 75 | μg |
| Chloride | 70 | mg | 70 | mg |
| Potassium | 80 | mg | 120 | mg |
| Boron | 150 | μg | 150 | μg |
| Nickel | 5 | μg | 5 | μg |
| Silicon | 2 | mg | 2 | mg |
| Vanadium | 10 | μg | 10 | μg |
| Lutein | 2400 | μg | 2600 | μg |
| Alpha Lipoic Acid | 60 | mg | 60 | mg |

### Example 7

### Animal Test

Diabetes was induced in female mice (body weight 180-220 g) with streptozotocin. The mice were fed with different substances to determine the effects of each on decreasing blood sugar level. The activity of G6PDH (glucose-6-phosphate dehydrogenase) and the blood sugar level were monitored. The mice were fed with Metaglip, the formulations for female as shown in Table 1 and Table 5, the placebo. The administration dosage is 10 times the dosage used on human (Metaglip: 1 mg/100 mg/Kg body weight, the formulation of the invention: 720 mg/Kg body weight). The blood samples were collected from the tail vein of the mice. Each group had 8 mice. Health mice were taken as the control.

The results were summarized in Table 7-1 and Table 7-2 (The values are the means of each group):

TABLE 7-1

| Level of blood sugar | | | | |
|---|---|---|---|---|
| (mg/L) | Initial value | 1^{st}. week | 2^{nd}. week | 4^{th}. week |
| Metaglip | 270.11 ± 18.42 | 170.11 ± 11.66 | 140.69 ± 9.44 | 88.83 ± 5.14 |
| Composition of | 268.83 ± 19.61 | 210.71 ± 16.11 | 170.38 ± 13.36 | 108.16 ± 5.89 |
| Table 1 | | | | |
| Composition of | 269.33 ± 20.16 | 168.31 ± 11.09 | 128.44 ± 9.19 | 85.51 ± 5.41 |
| Table 5 | | | | |
| Placebo | 269.97 ± 19.12 | 284.33 ± 19.92 | 331.11 ± 20.63 | 366.31 ± 21.79 |
| Health mice | 81.87 ± 4.92 | 83.87 ± 5.42 | 85.09 ± 5.72 | 83.63 ± 4.81 |

Two mice in the group of placebo died at the 9^{th} Week, and the data of the 10^{th}. week were not shown. The blood sugar level (mg/L, at the 10^{th}. week): Metaglip: 86.77 ± 5.01; Formulation of Table 1: 85.78 ± 5.09.

TABLE 7-2

| Activity of | | | | |
|---|---|---|---|---|
| G6PDH | Initial value | 1^{st}. week | 2^{nd}. week | 4^{th}. week |
| Metaglip | 51% | 59% | 66% | 65% |
| Composition of | 53% | 76% | 83% | 92% |
| Table 1 | | | | |
| Composition of | 49% | 71% | 80% | 91% |
| Table 5 | | | | |
| Placebo | 54% | 53% | 53% | 49% |
| Health mice | 100% | 100% | 100% | 100% |

Explanation:
1. The recovery formulation according to the invention (the formulation of Table 1), as measured the 10^{th} week, showed a capacity of decreasing blood sugar equivalent to that of Metaglip. And, the immediate-decrease formulation according to the invention (the composition of Table 5), from the start of the administration, showed a superior efficacy over Metaglip.
2. The formulations of the invention showed the best efficacy in recovering the activity of G6PDH. Over 90% of the activities of G6PDH were recovered by both formulations as measured at the 4^{th} week. The activity in the health mice was taken as 100%.

### Example 8

### Summary of Clinical Studies

Health conditions of five diabetes patients taking the recovery formulation of the invention were monitored for 9 months to 3 years. The dosage amount was in accordance to the amounts in Table 1, and the frequency was once a day.

8-1. Diabetes Patient JSJ:

JSJ was a 63 year-old female type II diabetes patient, who rarely did exercise, and ate normally. The monitoring included testing urine glucose with test papers by the patient and testing the fasting whole blood sugar every 6 months. JSJ had been diagnosed diabetes for 9 years before starting on the combined formulation of the invention. Before the study started, JSJ's urine glucose was measured to be “+++”, the fasting whole blood sugar was 14.3 mmol/L, and the urine ketone test showed negative.

JSJ used the combined formulation of the invention only, eating normally. Urine glucose test turned negative, and the fasting whole blood sugar went down to 7.1 mmol/L when she had used the combined formulation of the invention for 3 months. In the past 3 years on the formulation of the invention, JSJ's level of blood sugar was maintained within the normal range (5.8-7.1 mmol/L), and both the urine glucose and the urine ketone were negative in most of the tests. Only for one time, after a weeding feast, the urine glucose was measured to be “+++”, but went back to normal 3 days later. In the past 3 years, JSJ rarely complained the symptoms such as overdrinking, over-urinating, overeating, weight-lossing, dizziness, hypodynamia and the like. And JSJ has been enjoying her life like a health person.

8-2. Diabetes Patient DJ:

DJ was a 61 year-old female type II diabetes patient. She rarely did exercise, and ate normally. The monitoring included testing urine glucose with test papers by the patient and testing the fasting whole blood sugar every 6 months. DJ had been diagnosed diabetes for 11 years before starting on the combined formulation of the invention. Before the study started, DJ's urine glucose was measured to be “+++”, the fasting whole blood sugar was 13.9 mmol/L, and the test on urine ketone showed negative.

DJ used the vitamin complexes only in the first year. In that period, results of urine glucose was always “++” to “++++”. At the end of the first year, DJ complained severe symptoms such as dizziness, hypodynamia and the like with unknown causes. She was then hospitalized and examined. As measured, her blood sugar level was 18.6 mmol/L and the test on urine ketone showed positive. She was immediately treated with injection of insulin, and started on the combined formulation of the invention. 2 months later, both urine glucose test and the urine ketone test showed negative. 5 months later, the urine glucose test showed negative, and fasting whole blood sugar was 6.9 mmol/L. DJ have been persisting with the combined formulation of the invention from then on. In the 2 years, both the urine glucose test and the urine ketone test were always negative. DJ barely complained the symptoms such as overdrinking, over-urinating, overeating, weight-losing, dizziness, hypodynamia and the like. And SJS has been enjoying her life like a health person.

8-3. Diabetes Patient LXJ:

LXJ was a 45 year-old male type II diabetes patient. He went on jogging for at least 30 min at least three times a week, and ate normally. He did not show any chronic diabetic complications. The monitoring included testing urine glucose with test paper by the patient and testing the fasting whole blood sugar every 6 months. LXJ had been diagnosed diabetes for 3.5 years before he started to take the formulation of the invention. Before the study, LXJ's urine glucose in urine was measured to be “+++”, the fasting whole blood sugar was 13.3 mmol/L, and the test on urine ketone showed negative.

LXJ used the formulation of the invention only, eating normally. After using the formulation of the invention for 2.5 months, LXJ's urine glucose test turned negative, the fasting whole blood sugar went down to 8.1 mmol/L. In the next 5 months on the formulation of the invention, LXJ's level of blood sugar was maintained within the normal range. At the 8^{th}. month, the fasting whole blood sugar was measured to be 7.1 mmol/L, and the tests on urine glucose and urine ketone both showed negative. In the past 9 months, LXJ barely complained the symptoms such as overdrinking, over-urinating, overeating, weight-losing, dizziness, hypodynamia and the like. And LXJ has been enjoying her life like a health person.

8-4. Diabetes Patient ZXD:

ZXD was a 51 year-old female type II diabetes patient. She rarely did exercises, and ate normally. She did not show any chronic diabetic complications. The monitoring included testing urine glucose with test papers by the patient herself and testing the fasting whole blood sugar every 6 months. ZXD had been diagnosed diabetes for 6 years before she started to take the formulation of the invention. Before the study, ZXD's urine glucose was measured to be “+++”, the fasting whole blood sugar was 14.1 mmol/L, and the test on urine ketone showed negative.

After using the formulation of the invention for 3 months, ZXD's blood sugar level went down to normal, the fasting whole blood sugar was 7.8 mmol/L and the test on urine ketone showed negative. After 6 months, the test on urine glucose turned negative, and the level of blood sugar was 6.3 mmol/L. In the past 9 months, the tests on urine glucose and urine ketone were both negative. She barely complained the symptoms such as overdrinking, over-urinating, overeating, weight-losing, dizziness, hypodynamia and the like. And she has been enjoying her life like a health person.

8-5. Diabetes Patient LDW:

LDW was a 56 year-old male type II diabetes patient. He rarely did exercise, and ate normally. He complained the chronic diabetic complications such as slight fundus rentinosi, neuropathy-associated nausea and vomiting. The monitoring included testing urine glucose with test papers by the patient himself and testing the fasting whole blood sugar every 6 months. LDW had been diagnosed diabetes for 9 years before he started to take the formulation of the invention. Before the study, LDW's urine glucose was measured to be “+++”, the fasting whole blood sugar was 15.3 mmol/L, and the test on urine ketone showed positive.

LDW used the formulation of the invention only, eating normally. After LDW using the formulation of the invention for 4.5 months, the test on urine glucose turned negative, the fasting whole blood sugar went down to 8.4 mmol/L. After he using the formulation of the invention for 6 months, the level of blood sugar was maintained within the normal range (6.9 mmol/L, measured on the 6^{th}. month), and both the test on the urine glucose and the test on the urine ketone showed negative. The diabetic retinopathy was controlled, and the nausea and the vomiting caused by the diabetic neuropathy disappeared, no other symptoms of chronic diabetic complications arising.

1. A combined formulation for preventing and/or treating diabetes, comprising Vitamin E, chromium, selenium, lutein, folic acid, Vitamin C, alpha_lipoic acid, zinc, glutathione, lycopene, nicotinamide, L-arginine, vanadium and a pharmaceutical acceptable carrier.

3. The combined formulation of claim 1, further comprising coenzyme Q10, a vitamin complex and mineral.

4. The combined formulation of claim 1, further comprising Metformin or/and a sulfonylurea-based hypoglycemic agent such as Glimepiride, Glibenclamide or Glipizide as one or more additional hypoglycemic agents.

5. The combined formulation of claim 1, wherein, based on a unite dosage of the said formulation, the content of Vitamin E is 50-3000 IU, the content of chromium is 1.0-1000 μg, the content of selenium is 1.0-1000 μg, the content of lutein is 1.0-100 mg, the content of folic acid is 50-2000 μg, the content of Vitamin C is 10-2000 mg, the content of alpha_lipoic acid is 1.0-3000 mg, the content of zinc is 10-300 mg, the content of glutathione is 10-800 mg, the content of lycopene is 0.1-300 mg, the content of nicotinamide is 10-1800 mg, the content of L-arginine is 100-3000 mg, and the content of vanadium is 2-300 μg.

6. The combined formulation of claim 1, wherein, based on a unite dosage of the said formulation, the content of Vitamin E is 100-1200 IU, the content of chromium is 10-500 μg, the content of selenium is 10-680 μg, the content of lutein is 0.2-80 mg, the content of folic acid is 100-1000 μg, the content of Vitamin C is 50-800 mg, and the content of alpha alpha_lipoic acid acid is 5.0-1200 mg.

7. The combined formulation of claim 1, wherein, based on a unite dosage of the said formulation, the content of Vitamin E is 150-600 IU, the content of chromium is 50-380 μg, the content of selenium is 50-380 μg, the content of lutein is 0.5-30 mg, the content of folic acid is 200-900 μg, and the content of Vitamin C is 100-700 mg.

9. The combined formulation of claim 1, wherein, based on a unite dosage of the said formulation, the content of lycopene is 0.3-200 mg.

10. The combined formulation of claim 1, wherein, based on a unite dosage of the said formulation, the content of coenzyme Q10 is 40-400 mg.

11. The combined formulation of claim 3, wherein, based on a unite dosage of the said formulation, Glimepiride is present at an amount of 2-10 mg, or Glibenclamide is present at an amount of 2.5-15 mg, or Glipizide is present at an amount of 2.5-30 mg or Metformin is present at an amount of 200-1000 mg.
